(19) United States
(12) Patent Application Publication
(10) Pub. No.: US 2011/0081387 A1
(43) Pub. Date: Apr. 7, 2011

(54) LIPOSOMES ENCAPSULATING AN OXAZOLIDIN-2-ONE COMPOUND

(75) Inventors: Valérie ALARD, Orleans (FR); Angélique PICHOT, Saint Jean De Braye (FR); Thierry POUGET, Saint Cyr En Val (FR); Brigitte NOE, Orleans (FR)

(73) Assignee: LVMH RECHERCHE, Saint Jean De Braye (FR)

(21) Appl. No.: 12/700,072

(22) Filed: Feb. 4, 2010

(30) Foreign Application Priority Data
Oct. 6, 2009 (FR) 0956946

(51) Int. Cl. A61K 8/14; A61K 9/127; A61K 31/421; A61Q 19/00; A61Q 19/08

(52) U.S. Cl. 424/401; 424/450; 514/376

(57) ABSTRACT

The invention is directed to liposomes comprising at least one wall formed by a bilayer of amphiphilic lipids and containing an oxazolidin-2-one compound substituted in the 4-position, of formula (I):

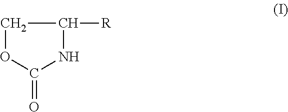

wherein the R represents a linear or branched, saturated or unsaturated hydrocarbon-based group containing from 4 to 30 carbon atoms. These liposomes may be used in the form of an aqueous dispersion in cosmetic or dermatological compositions comprising said dispersion; and at least one cosmetically or dermatologically acceptable excipient.

### CROSS REFERENCE TO RELATED APPLICATION

This application claims the benefit of French Patent Application No. 0956946, filed Oct. 6, 2009, the entirety of which is incorporated herein.

### TECHNICAL FIELD

The subject of the present invention is liposomes comprising an oxazolidin-2-one compound, compositions comprising said liposomes, and cosmetic methods using said compositions.

### BACKGROUND

Oxazolidin-2-one compounds substituted in the 4-position, of formula (I)

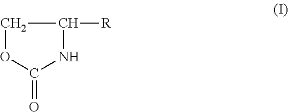

in which the substituent R represents a linear or branched, saturated or unsaturated hydrocarbon-based group, are known in the art. Some compounds corresponding to this formula have in particular been disclosed in Foglia et al.; J. Org. Chem., 1967, 32 (1), 75-78.

Oxazolidin-2-one compounds substituted in the 4-position are agents which improve crossing of the cutaneous barrier for hydrophilic active ingredients. U.S. Pat. No. 4,960,771 describes the use of oxazolidin-2-one derivatives of formula (I), including in particular 4-decyloxazolidin-2-one, as an agent for promoting transdermal penetration of an active ingredient contained in a composition for the treatment, in particular therapeutic treatment, of humans or animals.

FR 2908653 discloses the use of alkyloxazolidin-2-one compounds as moisturizing cosmetic agents.

None of these documents solves the technical problem comprising the provision of novel liposomes which improve the cutaneous penetration of cosmetic or dermatological agents.

### SUMMARY

The invention is directed to liposomes comprising at least one wall formed from a bilayer of amphiphilic lipids, which liposome contains an oxazolidin-2-one compound substituted in the 4-position, of formula (I):

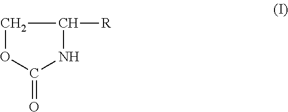

in which the substituent R represents a linear or branched, saturated or unsaturated hydrocarbon-based chain containing from 4 to 30 carbon atoms. Methods of making and using these liposomes is also described.

### DETAILED DESCRIPTION OF ILLUSTRATIVE EMBODIMENTS

The main objective of the present invention is to solve the technical problem comprising the provision of novel liposomes which improve the cutaneous penetration of cosmetic or dermatological agents.

The main objective of the present invention is also to solve the technical problem comprising the provision of novel cosmetic or dermatological formulations that may be in the form of an aqueous phase while at the same time containing a permeating agent that is insoluble in said phase, thus extending the use of this permeating agent to novel aqueous compositions.

The objective of the present invention is also to provide a simple solution to these technical problems, that is inexpensive and can be used on the industrial scale.

The applicant has discovered that, by encapsulating an oxazolidin-2-one compound substituted in the 4-position, of formula (I), as defined above, in liposomes according to the invention, it is possible to incorporate said compound into aqueous media, in which media it is normally insoluble owing to its highly lipophilic nature. The aqueous media containing this compound can then be used in the preparation of cosmetic or dermatological compositions.

It has been discovered that the liposomes containing an oxazolidin-2-one compound substituted in the 4-position, of formula (I), are stable in various forms of cosmetic or dermatological compositions.

Furthermore, it has been discovered that the liposomes containing an oxazolidin-2-one compound substituted in the 4-position have the additional advantage of significantly and unexpectedly promoting the penetration of cosmetic or dermatological active agents optionally present in said compositions, in a greater amount or to a greater depth in the superficial layers of the skin, thus solving the abovementioned technical problems.

### DEFINITIONS

In the description and the claims, the term “liposome” is intended to mean a microscopic corpuscle comprising at least one outer wall formed from a bilayer of amphiphilic lipids, and containing at least one aqueous compartment.

It is known that amphiphilic lipids, by virtue of their structure made up of a hydrophilic head connected to one or more lipophilic groups, become organized in bilayers in the presence of an aqueous medium, the interior of these bilayers forming a lipophilic internal compartment. Located inside this lipophilic compartment may be compounds which have a particular affinity for lipids.

Liposomes formed from a single lipid bilayer, usually called unilamellar liposomes, contain a single aqueous compartment, whereas liposomes with several walls, usually called multilamellar liposomes, contain several aqueous compartments located between the bilayers and at the core of said liposomes.

The liposomes and the methods for preparing them, in particular in the form of an aqueous dispersion, are known to those skilled in the art (reference may be made, for example, to the book “Liposomes as Tools in Basic Research and Industry”, Philippot J. and Schubert F., CRC Press Inc, 1995).

In the present description and the claims, the term “lipid” covers all substances comprising a “fatty” hydrocarbon-based chain containing at least 5 carbon atoms, in particular between 5 and 30 carbon atoms.

Similarly, for the purpose of the invention, the term “fatty alcohols” is intended to mean alcohols of which the carbon-based chain contains at least 5 carbon atoms, in particular between 5 and 30 carbon atoms.

The first subject of the present invention is thus liposomes comprising at least one wall formed from a bilayer of amphiphilic lipids, characterized in that said liposomes contain an oxazolidin-2-one compound substituted in the 4-position, of formula (I):

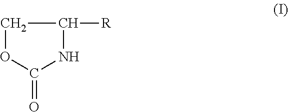

in which the substituent R represents a linear or branched, saturated or unsaturated hydrocarbon-based chain containing from 4 to 30 carbon atoms.

According to one preferred embodiment of the invention, the substituent R is an alkyl chain containing from 4 to 24 carbon atoms, advantageously from 4 to 18 carbon atoms.

According to one particularly preferred embodiment, the substituent R represents a decyl group, advantageously a linear decyl group, thus corresponding to 4-decyloxazolidin-2-one.

The amphiphilic lipid according to the invention has an ionic or nonionic hydrophilic group.

The amphiphilic lipid may be a phospholipid, such as phosphatidylcholine, phosphatidylethanolamine, phosphatidylserine, phosphatidylglycerol or phosphatidylinositol; a lecithin, a phosphoaminolipid; a sphingomyelin; a glycolipid such as a cerebroside and an alpha- or beta-glucoside of a fatty alcohol; a linear or branched polyglycerol ether; an ester of polyglycerol and of linear-chain fatty acids, such as an oxyethylenated polyglyceryl stearate; a polyoxyethylenated sterol.

According to one preferred embodiment of the invention, the wall of the liposomes according to the invention is formed by at least one lecithin or at least one lysolecithin, and more particularly at least one egg or soybean lecithin.

Advantageously, the lecithin is a soybean lecithin comprising at least 50% by weight of phosphatidylcholine, optionally as a mixture with a second soybean lecithin of different composition.

In one particularly preferred embodiment, the wall of the liposomes according to the invention is formed by a mixture of two soybean lecithins of different phospholipid composition, one being a mixture of phospholipids comprising more than 90% by weight of phosphatidylcholine, for example a soybean lecithin sold under the name Emulmetik® 930 by the company Lucas Meyer, the second being a mixture of phospholipids comprising between 15% and 30% of phosphatidylcholine, for example a soybean lecithin sold under the name Emulmetik® 300 IP by the company Lucas Meyer.

The liposomes of the invention are advantageously multilamellar liposomes preferably having an average diameter of approximately 150 to 200 μm, as measured by laser particle sizing in an aqueous suspension of liposomes or by transmission electron microscopy with preparation of the sample by cryofracture, starting from such a suspension.

According to one preferred embodiment, the liposomes according to the invention may also comprise other constituents, the nature of which may, for example, make it possible to improve the stability of the liposomes. By way of example, mention is made of the incorporation of sterols, for example cholesterol, into the lipid compartment of the bilayers in order to stiffen said bilayers. Also by way of example, it is possible to prepare liposomes that can be used in molecule-targeting techniques, by incorporating, into the walls of the liposomes, glycolipids which have an affinity for the target.

A second subject of the invention is directed toward an aqueous dispersion of liposomes according to the invention that can be used for the preparation of cosmetic or dermatological compositions.

A third subject of the invention is directed toward a cosmetic or dermatological composition comprising at least one cosmetically or dermatologically acceptable excipient, which composition comprises an aqueous phase in which liposomes as defined above are dispersed, or is obtained from the aqueous dispersion defined above.

The oxazolidin-2-one compound substituted in the 4-position, of formula (I), as defined above, may be incorporated into said composition at a concentration of between 0.05% and 5% by weight of the composition, in particular between 0.5% and 2% by weight.

The composition according to the invention preferably comprises at least one aqueous phase in which the liposomes of the invention are dispersed.

According to one particularly preferred embodiment, the composition is an aqueous composition, for example a lotion or a serum.

According to a second embodiment of the invention, the composition comprises a lipid phase, preferably a lipid discontinuous phase.

According to this embodiment, the composition is in particular an emulsion, preferably an oil-in-water emulsion.

Preferably, the dispersed lipid phase of the emulsion is barely apolar or is polar. It may in particular comprise or be constituted of triglycerides.

According to one particularly advantageous variant of the invention, the compositions of the invention may be in the form of a multiple emulsion in which the fatty phase of the emulsion is itself a water-in-oil (W/O) emulsion.

Preferably, the composition according to the invention also comprises at least one cosmetic or dermatological active agent, at least one part of which is advantageously encapsulated within the liposomes present in the composition.

The active agent is located in the hydrophilic or lipophilic compartments of the liposome, depending on its own affinity.

The active agent which has an affinity for lipids is thus preferentially located in the lipophilic intramembrane spaces of the lipid bilayers forming the wall of the liposome, whereas the hydrophilic active agent is located in the hydrophilic intermembrane spaces or the core of said liposomes.

The cosmetic active agent may be advantageously chosen from the group composed of substances which have a skin-depigmenting activity or a skin-lightening activity; of substances which have a slimming activity; of substances which have a moisturizing activity; of substances which have a calming, soothing or relaxing activity; of substances which have a cutaneous capillary circulation-stimulating activity for improving the radiance of the complexion, in particular of the face; of substances which have a sebum-regulating activity for the care of greasy skin; of substances for cleansing or purifying the skin; of substances which have a free-radical-scavenging activity or of substances which have an anti-aging activity.

The composition according to the invention may advantageously comprise several cosmetically active substances chosen from the same group or else chosen from groups of substances having a different cosmetic effect.

In addition, the composition may advantageously comprise one or more water-soluble polymers, in particular for stabilizing the liposomes according to the invention.

These water-soluble polymers may be of synthetic or natural origin, in particular of plant origin.

The water-soluble polymers may advantageously be chosen from thickening or gelling texturing agents, so as to adapt the viscosity of the compositions comprising the liposomes according to the invention.

The water-soluble polymers are advantageously chosen from tensioning agents capable of forming a film on the skin, so as to obtain a mechanical skin tension effect (“tensioning effect”).

The term “tensioning agent” is intended to mean a polymer or a blend of polymers, optionally combined with at least one plasticizer, forming on the skin a film that produces the desired mechanical effect (skin tension) while at the same time being well-tolerated by the user (comfort and lack of tautness).

According to one particularly preferred embodiment of the invention, the composition comprises one or more water-soluble polymers advantageously chosen from:
water-soluble polysaccharides, in particular from the group constituted of starch or a derivative thereof; a cellulose derivative, in particular carboxymethylcellulose, hydroxymethylcellulose, hydroxyethylcellulose, cellulose acetate, methylcellulose; a pectin; a gum, in particular xanthan gum, guar gum, an alginate, in particular an alkali metal alginate, and most particularly a sodium or potassium salt or an extract containing same, for example an algal extract; a dextran, a carrageenate, hyaluronic acid,
acrylic, vinyl or carboxyvinyl polymers or copolymers, or polyvinylpyrolidone, which are optionally crosslinked,
proteins and protein hydrolysates, in particular an extract of cereals, of legumes or of oil-yielding plants, such as an extract of corn, of rye, of wheat, of buckwheat, of sesame, of pea, of broad bean, of lentil, of soybean and of lupin,
polymers derived from chitin, in particular chitosan and derivatives thereof.

According to one preferred variant of the invention, the composition according to the invention comprises a hydrophilic tensioning agent comprising at least one water-soluble polymer as defined above, optionally combined with at least one plasticizer.

Examples of such hydrophilic tensioning agents that can be used for the invention are described in FR 2828810 in the name of the applicant, included in the present patent application by way of reference.

The plasticizer optionally present may be chosen from all the compounds capable of performing the desired function. This agent may be water-soluble or water-insoluble and may optionally be in the form of an aqueous dispersion.

In particular, mention may be made of, alone or as a mixture, the usual plasticizers, such as glycols and derivatives thereof, for instance diethylene glycol ethyl ether, diethylene glycol methyl ether, ethylene glycol ethyl ether, ethylene glycol butyl ether, propylene glycol methyl ether, propylene glycol phenyl ether, dipropylene glycol ethyl ether, dipropylene glycol butyl ether, tripropylene glycol butyl ether or tripropylene glycol methyl ether; glycerol esters; acid esters such as citrates, phthalates, adipates, carbonates, tartrates, phosphates, sebacates; oxyethylenated derivatives such as oxyethylenated oils, in particular plant oils, such as oxyethylenated castor oil, and oxyethylenated silicone oils; water-soluble polymers or polymers in an aqueous dispersion, having a low glass transition temperature, below 25° C., preferably below 15° C.

The amount of plasticizer is chosen by those skilled in the art on the basis of their general knowledge, so as to obtain a polymeric film having the desired mechanical properties, while at the same time conserving cosmetically acceptable properties for the composition.

Preferably, the tensioning agent has the property of fading out the wrinkles and fine lines at the surface of the skin immediately after application of the cosmetic composition according to the invention. The composition of the invention is then more particularly intended for application to the face and the neck.

In one particularly preferred embodiment, the composition according to the invention comprises at least one alkali metal alginate and at least a second water-soluble polysaccharide, in particular an alkali metal salt of carboxymethylcellulose, preferably sodium carboxymethylcellulose, and optionally a hydrophilic polymer chosen from the group constituted of polyvinylpyrrolidone and polyvinyl alcohol, and mixtures thereof.

The concentration of water-soluble polymers is chosen so as to effectively protect the bilayers of amphiphilic lipids or surfactants, and more particularly the liposomes, against degradation thereof under the effect of surfactants, present in the medium of the composition, and optionally adjusted so as to produce a mechanical tensioning effect after application of the composition to the skin.

Preferably, the total amount of water-soluble polymer(s) is between 0.1% and 10% by weight of the composition, preferably between 0.1% and 2% by weight.

In addition, the composition according to the invention may comprise one or more other water-soluble compounds, such as, for example, a C₆ or C₁₂ sugar or a polyol, advantageously chosen from glucose, sorbitol, sucrose, lactitol and glycerol or an ether or ester thereof or derivative thereof.

These water-soluble molecules are advantageously obtained from a plant extract, it being possible for said extract to itself be used in the composition.

The composition according to the invention may also comprise one or more other cosmetically acceptable excipients chosen from the group constituted of pigments, coloring agents, rheological agents, fragrances, sequestering agents, electrolytes, pH adjusters, antioxidants, preservatives, and mixtures thereof, texturing agents, antisun agents or sunscreens.

The cosmetic composition may be a skincare product or a makeup product for the skin and may be, for example, in the form of a serum, a lotion, an emulsion, a hydrogel, in particular a mask, a stick or a patch.

The present invention also relates to the use of the liposomes in any form, advantageously in the form of an aqueous dispersion as described above, as an agent for promoting skin permeation.

The present invention also relates to the method for preparing liposomes according to the invention, said method comprising, first of all, the preparation of an aqueous phase concentrated with respect to amphiphilic lipids comprising the amphiphilic lipids as defined above and the compound of formula (I), and then the preparation of an aqueous dispersion comprising said liposomes, from said aqueous phase concentrated with respect to amphiphilic lipids.

The method comprises first of all the preparation of an aqueous phase concentrated with respect to amphiphilic lipids, also comprising the compound of formula (I).

The preparation of said aqueous phase concentrated with respect to amphiphilic lipids may in particular comprise a first step in which the amphiphilic lipids and the compound of formula (I) are dissolved in a glycol or a mixture of glycols, in particular a mixture of glycerol and 1,3-butylene glycol, and then a second step in which water is added to the mixture prepared in the first step, in an amount sufficient to hydrate the phase obtained in the first step, and thus to obtain said aqueous phase concentrated with respect to amphiphilic lipids.

The aqueous phase concentrated with respect to amphiphilic lipids advantageously comprises less than 50% by weight of water, preferably less than 30% by weight of water.

Each of these steps advantageously comprises a period of homogenization using a suitable homogenizer.

The aqueous phase concentrated with respect to amphiphilic lipids that have been prepared may subsequently be diluted and then optionally strongly sheared, for example using a homogenizer, so as to form an aqueous dispersion of liposomes.

As disclosed above, the invention also relates to a stable dispersion of liposomes, that can in particular be used for the preparation of the aqueous phase of a composition, constituting another subject of the invention.

The aqueous dispersion of liposomes is advantageously stabilized by incorporation of an aqueous solution comprising at least one water-soluble polymer, in particular a water-soluble polysaccharide, into an aqueous phase concentrated with respect to amphiphilic lipids, and shearing of the mixture obtained, so as to form said aqueous dispersion.

The invention is also directed toward a method for preparing the abovementioned cosmetic or dermatological compositions, which comprises the preparation of an aqueous dispersion of liposomes, as defined above, preferably comprising a cosmetic or dermatological active agent, and then the preparation of said cosmetic or dermatological composition using said aqueous dispersion and at least one cosmetically or dermatologically acceptable excipient.

In the particular case where the composition is an emulsion, advantageously an oil-in-water emulsion, the method for preparing the composition according to the invention comprises the preparation of a stock emulsion stabilized with at least one surfactant, preferably a nonionic surfactant, and then the mixing of said stock emulsion with the aqueous dispersion comprising the liposomes, advantageously prepared according to the method described above.

According to this preparation method, the hydrophilic or water-soluble polymer may be, without distinction, brought into contact with the aqueous phase concentrated with respect to amphiphilic lipids, during the step for preparing the aqueous dispersion comprising the liposomes, or else be incorporated directly into the stock emulsion.

In the advantageous variant of the method described above, the aqueous dispersion of liposomes is diluted in an aqueous solution comprising at least one alkaline salt of an alginate, it being possible for said aqueous solution to be the aqueous continuous phase of the emulsion according to the invention.

The cosmetic or dermatological active agent optionally added may be outside the liposomes in the aqueous phase of the aqueous dispersion, or else be partially or totally encapsulated in the liposomes.

According to a final aspect, the invention is also directed toward a cosmetic care method, which comprises the topical application, to an area of the body concerned, of an effective amount of a cosmetic composition comprising at least one cosmetic active agent, as defined above or in the following description, so as to obtain the desired cosmetic effect.

Said care method may, for example, comprise the application of an effective amount of a cosmetic composition comprising at least one cosmetic active agent for maintaining or reinforcing the hydration state of the skin and/or for preventing or delaying the appearance of the signs of skin aging or slowing down the effects thereof, so as to obtain such a cosmetic effect.

More particularly, in this cosmetic care method, a cosmetic care intended to prevent or delay the appearance of the signs of skin aging or to slow down the effects thereof, in particular intended to tone up the skin, and/or to promote the reduction or the resorption of wrinkles, commonly termed anti-age or anti-aging effect, or else for a care intended to protect the skin against various stresses, is carried out.

It is clear for those skilled in the art that all the embodiments or variant embodiments of the invention in the context of the first aspect also apply to the second aspect and to the third aspect of the invention.

Those skilled in the art also understand that the invention thus defined solves the technical problems of the art, in a satisfactory, reliable and inexpensive manner that can be used on the industrial and cosmetic scale.

Other objectives, features and advantages of the invention will emerge clearly in the light of the explanatory description which will follow, made with reference to several exemplary embodiments and tests carried out in vitro and in vivo, which follow, given simply by way of illustration and which cannot in any way limit the scope of the invention.

Throughout the description, and in particular in the examples, the percentages are given by weight; the temperature is ambient temperature, i.e. 22° C., plus or minus 3° C.; the pressure is atmospheric pressure, unless otherwise indicated.

### EXAMPLES

Example 1

### Dispersion of Liposomes in Accordance with the Invention

The composition of the aqueous dispersion of liposomes is the following (% expressed by weight of the composition):

Phase A

| | |
|---|---|
| Emulmetik ® 300 IP | 4.2 |
| Emulmetik ® 930 | 4.2 |
| 4-decyloxazolidin-2-one | 16.8 |
| 1,3-butylene glycol | 8.4 |
| Plant glycerol | 8.4 |

Phase B

| | |
|---|---|
| Purified water | 16.8 |

Phase C

| | |
|---|---|
| Phenoxyethanol | 0.2 |
| Purified water | qs 100 |

Emulmetik® 300 IP and Emulmetik® 930 are two soybean lecithins of different composition, in particular in terms of their respective phosphatidylcholine contents, sold by the company Lucas Meyer.

Phase A is homogenized using a deflocculating paddle mixer (Rayneri) at 1000 rpm, for 1 hour.

Phase B is added and then the mixture is homogenized for 20 minutes under the same conditions as above, so as to hydrate phase A. The aqueous phase concentrated with respect to amphiphilic lipids, which in the present case contains approximately 28.5% by weight of water, is thus obtained.

Finally, phase C is added to the aqueous phase concentrated with respect to amphiphilic lipids obtained in the previous step, and the mixture is vigorously stirred using an Ultra Turrax® for 30 minutes at 15000 rpm, so as to produce the aqueous dispersion of liposomes.

The aqueous dispersion of liposomes thus prepared is used to prepare the cosmetic compositions according to examples 2 to 4.

### Example 2

### Serum Comprising Liposomes

A concentrated serum is prepared according to the following formula (% expressed by weight of the composition).

| | |
|---|---|
| Dispersion of liposomes according to example 1 | 12 |

Phase A

| | |
|---|---|
| Phenoxyethanol | 0.7 |
| Sodium hyaluronate | <0.1 |
| Carbomer | 0.5 |
| Tetrasodium EDTA powder | 0.2 |
| Sodium hydroxide | 0.2 |
| Ascorbic acid | <0.1 |
| Glycerol | 3.3 |
| 1,3-Butylene glycol | 2.0 |
| Methyl gluceth-20 | 1.8 |
| Alpha-tocopheryl acetate | <0.1 |
| Purified water | qs 100 |

An aqueous solution comprising the compounds of phase A is prepared.

The dispersion of liposomes is prepared in accordance with example 1.

The aqueous dispersion of liposomes is incorporated into phase A, with stirring, in an amount sufficient for the 4-decyloxazolidin-2-one to be at 2% by weight in the final composition.

The serum thus obtained comprises multilayer liposomes in the aqueous phase.

### Example 3

### Emulsion Comprising Liposomes

An oil-in-water emulsion in which the dispersed oily phase solubilizes the 4-decyloxazolidin-2-one (% expressed by weight of the composition) is prepared.

| | |
|---|---|
| Dispersion of liposomes according to example 1 | 12 |

Phase A

| | |
|---|---|
| Phenoxyethanol | 0.7 |
| Sodium hyaluronate | <0.1 |
| Carbomer | 0.25 |
| Pemulen ® TR-1 | 0.25 |
| Tetrasodium EDTA powder | 0.2 |
| Sodium hydroxide | 0.2 |
| Ascorbic acid | <0.1 |
| Glycerol | 3.3 |
| 1,3-Butylene glycol | 2.0 |
| Methyl gluceth-20 | 1.8 |
| Alpha-tocopheryl acetate | <0.1 |
| Purified water | qs 100 |

Phase B

| | |
|---|---|
| Glyceryl tricaprate/caprylate | 5.0 |

Pemulen® TR-1 is an acrylic acid/C₁₀-C₃₀ alkyl methacrylate copolymer sold by the company LUBRIZOL.

Phase A is prepared in accordance with example 2. The oil (phase B) is dispersed in phase A.

### Example 4

### Rich Cream

The composition has the following formula (% expressed by weight of the composition):

| | |
|---|---|
| Dispersion of liposomes according to example 1 | 6.1 |

Phase A

| | |
|---|---|
| Steareth 2 flakes (Brij ® 72 flakes) | 1.3 |
| Steareth 21 flakes (Brij ® 721P) | 2.2 |
| 95% cetyl alcohol | 1.2 |
| Stearyl alcohol | 1.2 |
| Stearic acid | 0.4 |
| Palmitic acid | 0.4 |
| Cetyl palmitate | 1.3 |
| Hydrogenated polyisobutene | 5.3 |
| Dicaprylyl carbonate | 4.5 |
| Caprylic/capric triglycerides | 5.0 |
| Dimethicone | 0.2 |
| Cyclopentasiloxane | 2.1 |
| Preservatives | 0.7 |

Phase B

| | |
|---|---|
| Glycerol | 3.5 |
| Purified water | 41 |
| Acrylates/C10-C30 alkyl acrylate crosspolymer | 0.5 |

Phase C

| | |
|---|---|
| Tetrasodium EDTA | 0.2 |
| Sodium hydroxide | 0.1 |
| Caprylyl glycol | 0.5 |
| Purified water | 4.8 |
| Sorbitol | 0.4 |
| Sodium alginate | 0.2 |
| Sodium carboxymethylcellulose | <0.1% |
| Polyvinyl alcohol | <0.1% |
| Vitamin E, sodium phosphate | 0.2 |
| Antioxidants | 0.2 |
| Purified water | qs 100 |

Phases A and B are heated to 85° C. separately so as to obtain two homogeneous solutions.

Phase B is then emulsified in oily phase A.

The oil-in-water (0/W) emulsion thus obtained is gradually cooled with stirring, and then the compounds of phase C are added at 70° C., in particular in order to neutralize the polymers.

The dispersion of liposomes, prepared in example 1, is added to the 0/W emulsion in an amount sufficient for the 4-decyloxazolidin-2-one to be at 1% by weight in the final composition.

The emulsion obtained comprises multilayer liposomes in the aqueous continuous phase. These liposomes are not destroyed by the action of the emulsion-stabilizing surfactants.

### Example 5

### Concentrated Night Serum

A concentrated serum is prepared according to the following formula (% expressed by weight of the composition).

| | |
|---|---|
| Dispersion of liposomes according to example 1 | 10 |

Phase A

| | |
|---|---|
| Glycerol | 1.8 |
| Phenoxyethanol | 0.6 |
| Purified water | 50 |

Phase B

| | |
|---|---|
| Glycerol | 0.3 |
| Xanthan gum | 0.3 |
| Citric acid•H₂O | <0.1 |
| Trisodium citrate•2H₂O | 0.5 |
| EDTA powder | 0.2 |
| Mg ascorbyl phosphate | 3.3 |
| Sepigel ® 305 (SEPPIC)* | 1.5 |
| Antioxidants, preservatives, fragrances | qs |
| Purified water | qs 100% |

*Sepigel ® 305: mixture of polyacrylamide, C₁₃-₁₄ isoparaffin and laureth-7.

An aqueous solution comprising the compounds of phase A is prepared, and the compounds of phase B are added thereto with stirring.

The dispersion of liposomes is then added with stirring and without incorporating air into the final composition.

### Example 6

### Study of Skin Permeability

Objective of the Study:

It is difficult for hydrophilic active agents to cross the stratum corneum, which is the most lipophilic cutaneous barrier, hence the advantage of vectorizing them so as to promote penetration thereof.

The objective of this study, carried out in vitro on frozen total pig ear skin, is to evaluate the advantage of encapsulating 4-decyloxazolidin-2-one, in order to improve the penetration and the distribution within the superficial layers of the skin of active agents that are unequally distributed in particular owing to their hydrophilicity.

1. Principle:

The study aims to evaluate the diffusion of a tracer, caffeine, through thawed pig ear skin, mounted on a Franz diffusion cell, under occlusive conditions.

Caffeine is chosen as the tracer owing to its hydrophilicity, which makes it relatively incapable of crossing the cutaneous barrier.

A Franz diffusion cell comprises two superimposed compartments which communicate through the membrane used for the study.

The skin, used as membrane, is placed, stratum corneum facing upward, between these two compartments. The aqueous solution or the galenical composition to be tested, containing caffeine, is introduced into the upper compartment in contact with the skin. A certain amount of caffeine, dissolved in the solution or the composition, crosses the membrane constituted of the skin, and is then collected in the lower compartment in a “receiver” solution.

The sampling device collects a sample of receiver solution at regular time intervals.

The samples are assayed by HPLC in order to determine the amount of caffeine having crossed the skin. Processing of the data makes it possible to calculate the caffeine fluxes, the penetration kinetics over 40 h, and also the 24 h absorption yields.

Various formulations are evaluated by this technique with the objective of identifying those which promote caffeine penetration in the skin.

2. Study Parameters:

Products Tested:

For the skin penetration study, a dispersion of liposomes is prepared according to example 1, other than the fact that the caffeine is added to phase C, at a percentage of 8.4% by weight of the dispersion.

The compositions according to the invention, in accordance with example 2 or 3, comprise an amount of liposomes such that the caffeine is present in the final composition at 1% by weight of the final composition.

“Lara Spiral” Franz Cell:

Exposure surface of 3.8 cm²

Receiver volume of 6.5 ml

Receiver Solution:

10 mmol phosphate buffer

120 mmol NaCl

2.7 mmol KCl

0.1% sodium azide

Surfactant: Tween 80® at 0.5%

Skin Quality:

Non-scalded pig ear skins, neither tattooed nor scratched, cut up into pieces of 3 cm by 3 cm and held taut at the surface of the lower compartment of the Franz cells.

Application Conditions:

Randomized deposit of each explant of 1 g of formulation, i.e. 10 mg of caffeine.

Number of Replicates:

4 Franz cells for the formulations comprising caffeine.

1 Franz cell for the control formulation without caffeine.

Temperature and Stirring:

Average temperature of the thermostated bath at 34.2° C.

Average stirring of 210 rpm

Gilson® Sampling Device

232 XL sample injector+control box+401C dilutor+Gilson® sampling needle.

Standard Range for Assaying Caffeine
Caffeine dissolved at from 0.1 to 500 ppm in a water/ethanol mixture (50/50)

3. Expression of the Results:

The amount of caffeine collected is expressed in μg/ml.

For each compartment studied, the proportion of caffeine absorbed is calculated on the basis of the amounts applied and of those collected.

The percentage of caffeine absorbed is determined as a function of time, in the lower compartment and in the skin through which it diffused.

4. Procedure:

At the end of the 40 of the study, the nonabsorbed excess of the compositions in the upper compartment is recovered, and the skin surface is washed with the receiver solution. The excess of formula recovered, the washing liquids and the upper part of the cell are immersed in ethanol/water (50/50) with stirring for 1 h in order to solubilize the active ingredient in the solvent.

The solution is diluted to 1/10^{th} in ethanol/water (50/50) and then filtered through 0.45 μm before assaying.

The epidermis and the dermis, after having been separated with a scalpel, are immersed separately for 24 h in an ethanol/water (50/50) solution and then stirred for half a day in order to extract the active ingredient.

The dermal and epidermal suspensions are stirred for 2 h and filtered through 0.45 μm before assaying by HPLC.

5. Results:

### 1) Caffeine Solubility Limit

In order to avoid any risk of underestimation of the cutaneous absorption, the operating conditions are fixed in such a way that the concentration of caffeine in the liquid of the receiver compartment does not exceed 10% of the limiting solubility concentration (16 mg/ml), i.e. 1.6 mg/ml.

2) Results

2. 1) Test 1

The compositions tested are sera.

A serum comprising liposomes, prepared according to example 2 (C1), is here compared with a control composed of a 1% by weight aqueous solution of caffeine (A1) and with the same serum without liposomes, but comprising 2% of 4-decyloxazolidin-2-one dispersed in the aqueous phase (B1).

The results are reported in table 1 below (% expressed relative to the initial amount of caffeine introduced into the upper compartment of the Franz cell):

TABLE 1

| | A1 | | C1 (serum |
|---|---|---|---|
| | (control | B1 | according to |
| | solution) | (control serum) | the invention) |
| 4-decyloxazolidin-2-one | − | + | + |
| liposomes | − | − | + |
| % at 40 h | 10.7 | 23.8 | 33.2 |

It results from table 1 that the encapsulation of 4-decyloxazolidin-2-one in liposomes makes it possible to obtain a significantly greater amount of caffeine having passed through the skin than that measured for the control formulations A1 and B1.

2. 2) Test 2

The compositions tested are emulsions.

The oily phase (phase B) is intended to solubilize the 4-decyloxazolidin-2-one. The emulsion comprising liposomes and prepared according to example 3 (B2) is compared with the same emulsion without liposomes, in which the 4-decyloxazolidin-2-one is simply solubilized in the oily phase (A2).

The results are reported in table 2 below (% expressed relative to the initial amount of caffeine introduced into the upper compartment of the Franz cell):

TABLE 2

| | A2 | B2 (emulsion |
|---|---|---|
| | (control) | according to the invention) |
| 4-decyloxazolidin-2-one | + | + |
| liposomes | − | + |
| % at 40 h | 14.2 | 28.3 |
| % in the skin | 3.5 | 3.9 |
| (dermis and epidermis) | | |

It results from table 2 that the encapsulation of 4-decyloxazolidin-2-one in liposomes enables a significantly greater amount of caffeine to pass through the skin than that measured for the control composition (A2) in which the 4-decyloxazolidin-2-one is solubilized in the dispersed oily phase.

2. 4) Test 3

A serum according to example 2, in which the content of 4-decyloxazolidin-2-one is varied from 0 (control) to 2%, is prepared.

The results are reported in table 3 below (% expressed relative to the initial amount of caffeine introduced into the upper compartment of the Franz cell):

TABLE 3

| | |
|---|---|
| | Serum according |
| Control | to example 2 |

| | | | |
|---|---|---|---|
| 4-decyloxazolidin-2-one | − | 0.5 | 2% |
| liposomes | + | + | + |
| % at 40 h | 18.6 | 34.5 | 42.7 |
| % in the skin | 5.0 | 6.3 | 6.5 |
| (dermis and epidermis) | | | |

The above test is reproduced, with the 4-decyloxazolidin-2-one content being substantially reduced, against the same control.

The results are reported in table 4 below (% expressed relative to the initial amount of caffeine introduced into the upper compartment of the Franz cell):

TABLE 4

| | |
|---|---|
| | Serum according |
| Control | to example 2 |

| | | | |
|---|---|---|---|
| 4-decyloxazolidin-2-one | − | 0.05 | 0.1% |
| liposomes | + | + | + |
| % at 40 h | 11.1 | 18.0 | 16.6 |
| % in the skin | 3.9 | 4.5 | 2.6 |
| (dermis and epidermis) | | | |

It results from tables 3 and 4 that the encapsulation of 4-decyl-oxazolidin-2-one significantly improves the skin penetration of caffeine at 4-decyl-oxazolidin-2-one concentrations that may vary by a factor of 40, which is completely unexpected for those skilled in the art.

What is claimed:

1. A liposome comprising at least one wall formed from a bilayer of at least one amphiphilic lipid, the liposome containing a compound of formula (I): 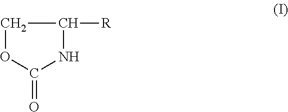
wherein R is a linear or branched, saturated or unsaturated hydrocarbon-based chain containing from 4 to 30 carbon atoms.

2. The liposome as claimed in claim 1, wherein R is an alkyl chain containing from 4 to 24 carbon atoms.

3. The liposome as claimed in claim 1, wherein R is a decyl group.

4. The liposome as claimed in claim 1, wherein the amphiphilic lipid has an ionic or nonionic hydrophilic group.

5. The liposome as claimed in claim 1, wherein the amphiphilic lipid is selected from the group consisting of a phospholipid, a phosphatidylcholine, a phosphatidylethanolamine, a phosphatidylserine, a phosphatidylglycerol, a phosphatidylinositol; a lecithin; a phosphoaminolipid; a sphingomyelin, a glycolipid, a cerebroside, an alpha-glucoside of a fatty alcohol, a beta-glucoside of a fatty alcohol, a linear or branched polyglycerol ether, an ester of polyglycerol, an ester of linear-chain fatty acids, an oxyethylenated polyglyceryl stearate, a polyoxyethylenated sterol, and a mixture thereof.

6. The liposome as claimed in claim 1, wherein the wall of said liposome is formed from at least one lecithin or at least one lysolecithin.

7. The liposome as claimed in claim 6, wherein said wall is formed by a mixture of two soybean lecithins of different phospholipid composition, one being a mixture of phospholipids comprising more than 90% by weight of phosphatidylcholine, the second being a mixture of phospholipids comprising between 15 and 30% of phosphatidylcholine.

8. The liposome as claimed in claim 1, wherein said liposome is a multilamellar liposome.

9. An aqueous dispersion of liposomes as claimed in claim 1.

10. A cosmetic or dermatological composition comprising at least one cosmetically or dermatologically acceptable excipient, the composition comprising an aqueous phase in which liposomes as defined in claim 1 are dispersed or an aqueous dispersion of liposomes as defined in claim 1.

11. The composition as claimed in claim 10, wherein the compound of formula (I) is incorporated into said composition at a concentration of between 0.05% and 5% by weight of the composition.

12. The composition as claimed in claim 10, wherein said composition comprises at least one aqueous phase in which the liposomes are dispersed.

13. The composition as claimed in claim 12, wherein the composition is an aqueous composition.

14. The composition as claimed in claim 12, wherein the composition is a lotion or a serum.

15. The composition as claimed claim 10, wherein said composition comprises a lipid phase.

16. The composition as claimed in claim 15, wherein said liposome comprises a dispersed lipid phase and wherein said dispersed lipid phase comprises or is constituted of triglycerides.

17. The composition as claimed in claim 15, wherein said composition is an emulsion.

18. The composition as claimed in claim 10, wherein said composition also comprises at least one cosmetic or dermatological active agent, wherein at least a part of which is encapsulated within the liposomes present in the composition.

19. The composition as claimed in claim 18, wherein said active agent is selected from the group consisting of a substance which has a skin-depigmenting activity or a skin-lightening activity; a substance which has a slimming activity; a substance which has a moisturizing activity; a substance which has a calming, soothing or relaxing activity; a substance which has a cutaneous capillary circulation-stimulating activity for improving the radiance of the complexion; a substance which has a sebum-regulating activity for the care of greasy skin; a substance for cleansing or purifying the skin; a substance which has a free-radical-scavenging activity, a substance which has an anti-aging activity, and any mixture thereof.

20. The composition as claimed in claim 10, wherein said composition comprises one or more water-soluble polymers that stabilize liposomes selected from the group consisting of:
water-soluble polysaccharides, starch or a derivative thereof; a cellulose derivative, carboxymethylcellulose, hydroxymethylcellulose, hydroxyethyl-cellulose, cellulose acetate, methylcellulose; a pectin; a gum, xanthan gum, guar gum, an alginate, an alkali metal alginate, a sodium or potassium salt or an extract containing same, an algal extract; a dextran, a carrageenate, hyaluronic acid;
acrylic, vinyl polymers or copolymers, carboxyvinyl polymers or copolymers, polyvinylpyrolidone, optionally crosslinked,
proteins, protein hydrolysates, an extract of cereals, an extract of legumes, an extract of oil-yielding plants, an extract of corn, an extract of rye, an extract of wheat, an extract of buckwheat, an extract of sesame, an extract of pea, an extract of broad bean, an extract of lentil, an extract of soybean, an extract of lupin,
polymers derived from chitin, chitosan and derivatives thereof,
and a mixture thereof.

21. The composition as claimed in claim 10, wherein said composition comprises a hydrophilic tensioning agent comprising at least one water-soluble polymer as defined in claim 20, optionally combined with a least one plasticizer.

22. The composition as claimed in claim 20, wherein said composition comprises at least one alkali metal alginate and at least a second water-soluble polysaccharide.

23. The composition as claimed in claim 20, wherein the total amount of water-soluble polymer(s) is between 0.1% and 10% by weight of the composition.

24. The composition as claimed in claim 10, wherein said composition comprises one or more other water-soluble compounds selected from the group consisting of a C₆ or C₁₂ sugar, a polyol, glucose, sorbitol, sucrose, Lactitol, glycerol, an ether or ester thereof or derivative thereof, optionally obtained from a plant extract.

25. The composition according to claim 10, wherein said composition comprises one or more other cosmetically acceptable excipients selected from the group consisting pigments, coloring agents, rheological agents, fragrances, sequestering agents, electrolytes, pH adjusters, antioxidants, preservatives, and mixtures thereof, texturing agents, antisun agents, and sunscreens.

26. The composition as claimed in claim 10, wherein said composition is a skin care product or a makeup product for the skin, and is in the form of a serum, a lotion, an emulsion, a hydrogel, a mask, a stick, or a patch.

27. A method for promoting skin permeation, wherein said method comprises the topical application of the liposomes as claimed in claim 1 or an aqueous dispersion of liposomes as claimed in claim 1.

28. A method for preparing liposomes as claimed in claim 1, said method comprising
preparing an aqueous phase concentrated with respect to amphiphilic lipids comprising a compound of formula (I); and
preparing an aqueous dispersion comprising said liposomes, from said aqueous phase concentrated with respect to amphiphilic lipids.

29. The method as claimed in claim 28, wherein the preparation of said aqueous phase concentrated with respect to amphiphilic lipids comprises
a first step comprising dissolving the amphiphilic lipids and the compound of formula (I) in a glycol or a mixture of glycols, and
a second step comprising adding water to the mixture prepared in the first step in an amount sufficient to hydrate the phase obtained in the first step, and thus to obtain said aqueous phase concentrated with respect to amphiphilic lipids,
wherein the first and second steps comprise a period of homogenization using a mixer.

30. The method as claimed in claim 28, wherein the aqueous phase concentrated with respect to amphiphilic lipids comprises less than 50% by weight of water.

31. The method as claimed in claim 28, wherein the aqueous phase concentrated with respect to amphiphilic lipids that has been concentrated is then optionally strongly sheared, so as to form the aqueous dispersion of liposomes.

32. The method as claimed in claim 28, wherein the aqueous dispersion of liposomes is stabilized by incorporating an aqueous solution comprising at least one hydrophilic or water-soluble polymer into the aqueous phase concentrated with respect to amphiphilic lipids, and shearing of the mixture obtained, so as to form said aqueous dispersion.

33. A method for preparing a cosmetic or dermatological composition comprising an aqueous dispersion as claimed in claim 9, said aqueous dispersion optionally comprising a cosmetic or dermatological active agent, wherein said method comprising admixing said aqueous dispersion and at least one cosmetically or dermatologically acceptable excipient for preparing said cosmetic or dermatological composition.

34. The method as claimed in claim 33, wherein said composition is an emulsion, and which method comprises the following steps:
preparing a stock emulsion stabilized with at least one surfactant, and
mixing said stock emulsion with the aqueous dispersion as claimed in claim 9.

35. A method of cosmetic care comprising
applying topically, to an area of the body, an effective amount of a cosmetic composition as claimed in claim 18, so as to obtain a desired cosmetic effect.

36. The method of cosmetic care as claimed in claim 35, wherein the method comprises
applying an effective amount of a cosmetic composition comprising at least one cosmetic active agent for maintaining or reinforcing the hydration state of the skin or for preventing or delaying the appearance of the signs of skin aging or for slowing down the effects thereof, so as to obtain a cosmetic effect.

37. The method of cosmetic care as claimed in claim 35, wherein said cosmetic care is for preventing or slowing down the appearance of the signs of skin aging or slowing down the effects thereof, toning up the skin, promoting the reduction or the resorption of wrinkles, or for protecting the skin against various stresses.
